(19) United States
(12) Patent Application Publication
(10) Pub. No.: US 2022/0404354 A1
(43) Pub. Date: Dec. 22, 2022

(54) REAL-TIME, POINT OF CARE DIAGNOSTIC AND METHOD OF USE THEREOF

(71) Applicant: Quantum Materials Corporation, San Marcos, TX (US)

(72) Inventors: Andrew ROBINSON, San Marcos, TX (US); Krishna KOWLGI, San Marcos, TX (US); Stephen SQUIRES, San Marcos, TX (US); Brent Wade FERGUSON, San Marcos, TX (US); Nathanael J. BARREE, San Marcos, TX (US)

(21) Appl. No.: 17/738,289

(22) Filed: May 6, 2022

Related U.S. Application Data
(60) Provisional application No. 63/185,836, filed on May 7, 2021.

(51) Int. Cl. G01N 33/558; G01N 21/84; G01N 21/78; B82Y 20/00; G01N 33/569

(52) U.S. Cl. CPC G01N 33/558; G01N 21/8483; G01N 21/78; B82Y 20/00; G01N 33/56983; G01N 2333/165

(57) ABSTRACT

The invention encompasses a real time, point of care, diagnostic system, including a lateral flow test cassette, a data reader, and an application for processing test results and methods of diagnosis of a disease or virus.

### CROSS-REFERENCE TO RELATED APPLICATION

This application claims the benefit of and priority to U.S. Provisional Application No. 63/185,836 filed May 7, 2021

, this disclosure is hereby incorporated by reference in its entirety.

### FIELD

The invention encompasses a real time, point of care, diagnostic system, including a lateral flow test cassette, a data reader, and an application for processing test results and methods of diagnosis of a disease or virus.

### BACKGROUND

Existing lateral flow immunochromatographic assays (LFIAs) have limitations in performance ang readability, among other things. FIG. 1 provides an overview comparison of different diagnostic methods. For example, LFIAs are limited in sensitivity, specificity and have problems with accuracy. Therefore, they are inferior to RT-PCR tests which are more reliable but are slow (requiring a day or more to get a result), and require intensive labor, instrumentation and are not suitable as point-of-care devices. In addition, LFIAs currently in the market predominantly use colloidal gold (CG) for the bio-labels. CG based LFIAs have low luminosity (quantum yields <1%), which makes reading of test lines difficult, and the test lines grow more faint with time. It is also very difficult to implement multiple analyte testing (multiplexing) as CG has a limited color gamut (red and purple). Moreover, CG have problems with manufacturing consistency and low shelf life, which make the LFIAs unreliable. Finally, CG based LFIAs are expensive to make due to the high price of the raw materials for gold.

### DESCRIPTION OF DRAWINGS

FIG. 1 is an overview comparison of different diagnostic methods.

FIG. 2A is an illustrative example of a smartphone with an exemplary data reader and the packaging and components of a lateral flow assay with test cassette and an exemplary test strip. FIG. 2B illustrates a lateral flow test cassette, a data reader, and an exemplary connection of the data reader with a smart phone or tablet.

FIG. 3 is an illustration of the components of an example implementation of the data reader.

FIG. 4 is a table describing differences between colloidal gold, quantum dots, and metal nanocluster biolabels.

FIG. 5A-5E are a representation of a test strip illustrating the principle of operation of one implementation of the diagnostic system.

FIG. 6 is an illustration of a lateral flow test cassette and test strip.

FIG. 7 is a depiction of filtered images and images detected by a smartphone when quantum dots are or are not detected at a control line and on a test line.

FIGS. 8A and 8B are a flow diagram of the process of using the app.

FIG. 9 is a depiction of different configurations of test strips implementing a covert testing capability.

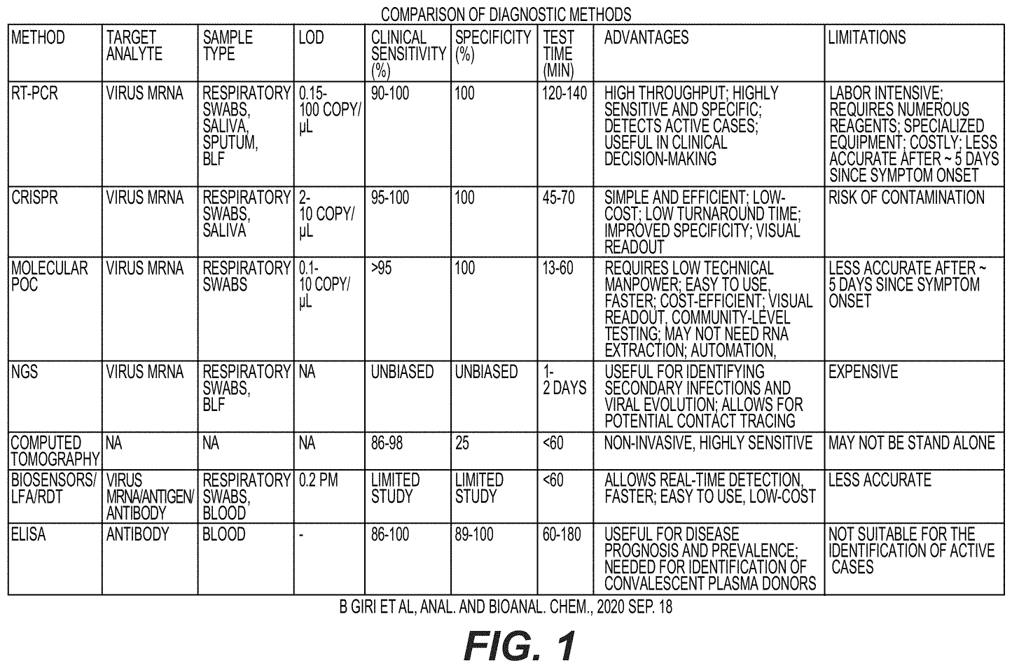

### DETAILED DESCRIPTION

The invention encompasses a real-time, lateral flow assay test that include one or more labels and systems and methods for reading such test that provide improved detection of different capture regions on the test strips, improved assay testing speed, and improved assay measurement sensitivity.

The term “lateral flow assay test strip” encompasses both competitive and non-competitive types of lateral flow assay test strips. A lateral flow assay test strip generally includes a sample receiving zone and a detection zone, and may or may not have a labeling zone. In some implementations, a lateral flow assay test strip includes a sample receiving zone that is located vertically above a labeling zone, and additionally includes a detection zone that is located laterally downstream of the labeling zone.

The term “analyte” refers to a substance that can be assayed by the test strip. Examples of different types of analytes include organic compounds (e.g., proteins and amino acids), hormones, metabolites, antibodies, pathogen-derived antigens, drugs, toxins, and microorganisms (e.g., bacteria and viruses).

As used herein the term “label” refers to a substance that has specific binding affinity for an analyte and that has a detectable characteristic feature that can be distinguished from other elements of the test strip. The label may include a combination of a labeling substance (e.g., a fluorescent particle, such as a quantum dot or a quantum dot on a bead) that provides the detectable characteristic feature and a probe substance (e.g., an immunoglobulin) that provides the specific binding affinity for the analyte. In some implementations, the labels have distinctive optical properties, such as luminescence (e.g., fluorescence) or reflective properties, which allow regions of the test strip containing different labels to be distinguished from one another.

The term “reagent” refers to a substance that reacts chemically or biologically with a target substance, such as a label or an analyte.

The term “capture region” refers to a region on a test strip that includes one or more immobilized reagents.

The term “test region” refers to a capture region containing an immobilized reagent with a specific binding affinity for an analyte.

The term “control region” refers to a capture region containing an immobilized reagent with a specific binding affinity for a label.

In certain embodiments, the diagnostic test system includes a housing, a reader, a data analyzer, and a memory. The housing includes a port for receiving a test strip. When the test strip is loaded in the port, the reader obtains light intensity measurements from the test strip. In general, the light intensity measurements may be unfiltered or they may be filtered in terms of at least one of wavelength and polarization. The data analyzer computes at least one parameter from one or more of the light intensity measurements. A results indicator provides an indication of one or more of the results of an assay of the test strip. In some implementations, the diagnostic test system is fabricated from relatively inexpensive components enabling it to be used for disposable or single-use applications.

The housing may be made of any one of a wide variety of materials, including plastic and metal. The housing forms a protective enclosure for the reader, the data analyzer, the power supply, and other components of the diagnostic test system. The housing also defines a receptacle that mechanically registers the test strip with respect to the reader. The receptacle may be designed to receive any one of a wide variety of different types of test strips.

In various embodiments, each of the test strips is a non-competitive type of lateral flow assay test strip that supports lateral flow of a fluid sample along a lateral flow direction and includes a labeling zone containing a labeling substance that binds a label to a target analyte and a detection zone that includes at least one test region containing an immobilized substance that binds the target analyte. One or more areas of the detection zone, including at least a portion of the test region, are exposed for optical inspection by the reader. The exposed areas of the detection zone may or may not be covered by an optically transparent window.

In other embodiments, the test strips are competitive type of lateral flow assay test strips in which the concentrations of the label in the test region decreases with increasing concentration of the target analyte in the fluid sample. Some of these embodiments include a labeling zone, whereas others of these implementations do not include a labeling zone.

Some of these competitive lateral flow assay test strip embodiments include a labeling zone that contains a label that specifically binds target analytes in the fluid sample, and a test region that contains immobilized target analytes as opposed to immobilized test reagents (e.g., antibodies) that specifically bind any non-bound labels in the fluid sample. In operation, the test region will be labeled when there is no analyte present in the fluid sample. However, if target analytes are present in the fluid sample, the fluid sample analytes saturate the label's binding sites in the labeling zone, well before the label flows to the test region. Consequently, when the label flows through the test region, there are no binding sites remaining on the label, so the label passes by and the test region remains unlabeled.

In other competitive lateral flow assay test strip embodiments, the labeling zone contains only pre-labeled analytes (e.g., gold adhered to analyte) and the test region contains immobilized test reagents with an affinity for the analyte. In these embodiments, if the fluid sample contains unlabeled analyte in a concentration that is large compared to the concentration of the pre-labeled analyte in the labeling zone, then label concentration in the test region will appear proportionately reduced.

The reader includes one or more optoelectronic components for optically inspecting the exposed areas of the detection zone of the test strip. In some implementations, the reader includes at least one light source and at least one light detector. In some implementations, the light source may include a semiconductor light-emitting diode and the light detector may include a semiconductor photodiode. Depending on the nature of the label that is used by the test strip, the light source may be designed to emit light within a particular wavelength range or light with a particular polarization. For example, if the label is a fluorescent label, such as a quantum dot, the light source would be designed to illuminate the exposed areas of the detection zone of the test strip with light in a wavelength range that induces fluorescent emission from the label. Similarly, the light detector may be designed to selectively capture light from the exposed areas of the detection zone. For example, if the label is a fluorescent label, the light detector would be designed to selectively capture light within the wavelength range of the fluorescent light emitted by the label or with light of a particular polarization. On the other hand, if the label is a reflective-type label, the light detector would be designed to selectively capture light within the wavelength range of the light emitted by the light source. To these ends, the light detector may include one or more optical filters that define the wavelength ranges or polarizations axes of the captured light.

The data analyzer processes the light intensity measurements that are obtained by the reader. In general, the data analyzer may be implemented in any computing or processing environment, including in digital electronic circuitry or in computer hardware, firmware, or software. In some embodiments, the data analyzer includes a processor (e.g., a microcontroller, a microprocessor, or ASIC) and an analog-to-digital converter. In the illustrated embodiment, the data analyzer is incorporated within the housing of the diagnostic test system. In other embodiments, the data analyzer is located in a separate device, such as a computer, that may communicate with the diagnostic test system over a wired or wireless connection.

In accordance with aspects described in this specification, a diagnostic system includes a multiplexed lateral flow test cassette, a data reader, and a smart phone or tablet. The multiplexed lateral flow test cassette comprises a lateral flow immunochromatographic assay and can include a housing, a test strip, and a QR code or another identifier, which may be readable by a sensor, such as an optical sensor on a smartphone or other device. The lateral flow immunochromatographic assay is a biochemical test that measures (qualitatively/quantitatively) the presence of analyte molecules (such as the proteins on a SARS-CoV-2 virus) with the help of sensory molecules (based on quantum dots/nanoparticles/metal nanoclusters) on a platform (basically a membrane that controls the transport of the molecules) that displays a visual pattern (e.g., lines, circles, arrows, or dotted lines) when a successful test run occurs. This visual pattern on the test strip in combination with a machine-readable code (e.g., a QR code or similar code) that coated on the assay housing (and packaging) is used to determine the result of the diagnosis. The diagnostic system can also include accessories, including a fluid vial, a test swab, and a PBS buffer.

The data reader includes a data and power connector and a camera sensor (e.g., using a CCD or CMOS sensor) inside a body of the data reader. A light source (e.g., LEDs) is integrated with the camera sensor for illuminating the test cassette to enable the camera sensor to detect test results from the test strip of the lateral flow test cassette. In example implementations, the light source may emit ultraviolet light (e.g., around 350 nm wavelength) or visible light (e.g., around 400 nm wavelength). The data reader holds the test cassette in place and uses a combination of the camera sensor and light source to activate and read the spectral signature of the visual pattern on the test strip (e.g., quantum dots) and to read the machine-readable code. To hold the test cassette in place, the data reader can include a slot into which the test cassette or test strip can be inserted and which appropriately positions the test cassette so that the camera sensor can detect the visual pattern on the test strip. The use of a power connector avoids the need for battery power, which can help reduce the cost of the data reader and, along with using biodegradable housing materials, make the data reader more environmentally friendly and disposable.

The data reader communicates with a smartphone or other computing device. For example, the data reader can be connected to the charging port of a smartphone or tablet. The smartphone or tablet can both power the data reader and provide a communications channel. An app on the smartphone or tablet provides instructions for a human tester to perform the test, receives data from the reader and processes the data. The app can also instruct the tester to redo the test if the test is invalid or inconclusive, issue the test result, and provide further instructions if needed. The system can be used in a point-of-care scenario (e.g., administered by a medical professional) or as a home-test kit (e.g., not administered by a third party but rather self-administered). In some implementations, the system can do quantitative analysis to find the amount of analyte in the test strip using, for example, an advanced reader (an intense UV light source and a high resolution CMOS sensor), a lateral flow test cassette adapted for high resolution image analysis (e.g., using materials that limit auto-fluorescence and have more exposed surface for analysis), and advanced image analysis software.

In some implementations, the system can be implemented with a reader and/or smartphone that is more economical, lightweight, and easier to use for self-testing applications. Such an implementation may provide qualitative results, use visible light (e.g., 400 nm wavelength), and rely upon a sensor that is widely available (e.g., 720 p Galaxy Core GC0308 with fixed focus). In alternative implementations, the system can be implemented with components that are more suitable for point-of-care applications because, for example, they may need a medical professional to operate or may be less economical. These more complex implementations may provide quantitative results, which may be facilitated using a lateral flow cassette design where the area from the conjugate pad to the absorbent pad is exposed to the reader for quantitative analysis. The reader module may incorporate intense ultraviolet lighting (e.g., 350 nm wavelength) and a better CMOS sensor (e.g., a 16 MP CMOS, such as a Sony IMX214 or better with autofocus), And the smartphone app may be capable of performing quantitative image analysis.

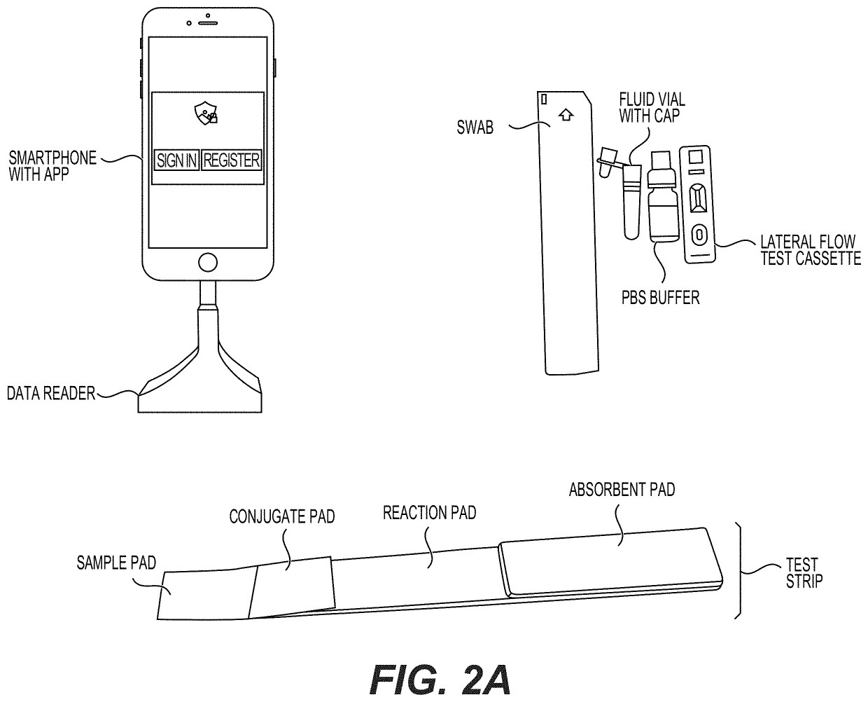

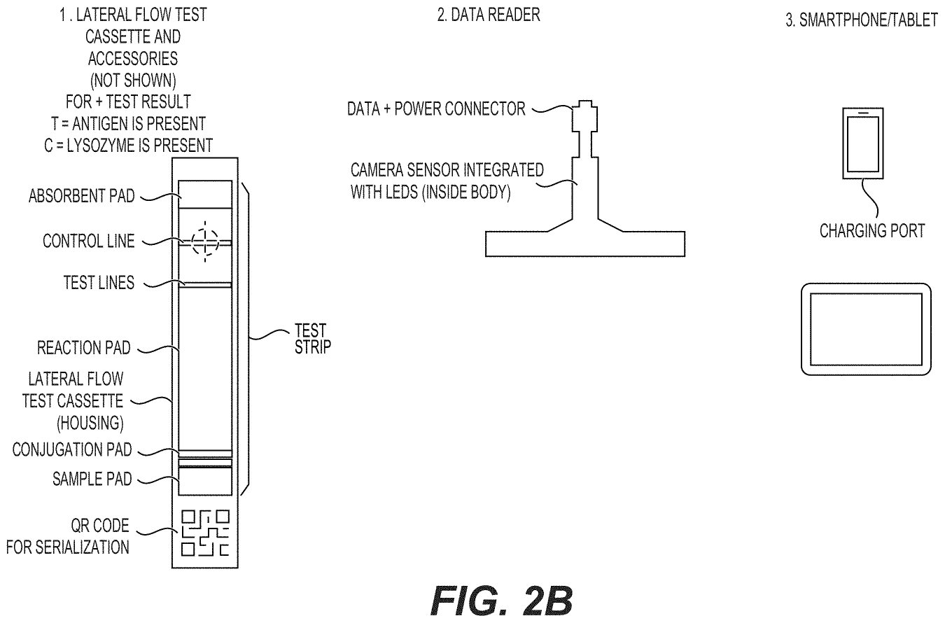

FIGS. 2A and 2B depict illustrative examples of a smartphone with the app installed and a data reader that connects to the smartphone using the data and charging port of the smartphone. A test kit may include a lateral flow test cassette, a swab, a fluid vial with cap, and a buffer (e.g., PBS (phosphate buffered saline) buffer) or extraction fluid for use in performing a test. The lateral flow test cassette includes a housing that fits the reader in one orientation and prevents usage of fake cassettes. The test strip inside of the lateral flow test cassette housing includes a sample pad (e.g., where a saliva sample is placed), a conjugation pad (e.g., containing antibody-nanoparticle conjugates), a reaction pad (e.g., where test result lines and a control line appear, and an absorbent pad. When a saliva sample is placed on the sample pad, the sample flows along the test strep passing through the conjugate pad into the nitrocellulose membrane, which includes test and control lines, and then to the absorbent pad, which helps with the flow of the sample across the test strip. The test lines appear when a specific antigen is present in the sample, and the control line appears when amylase or other enzyme or matrix loading control is present. The sample pad can use standard glass fiber/cellulose material, the conjugate pad uses unique antibodies and biolabels as described in this specification, the reaction pad can use a standard membrane (e.g., produced by Sartorius/GE), and the absorbent pad can use standard glass fiber/cellulose. The lateral flow test cassette also includes a QR code that can be used to serialize data for uniquely identifying the test kit, identifying the type of test, and/or decoding what specific spectral patterns represent if they appear on the reaction pad.

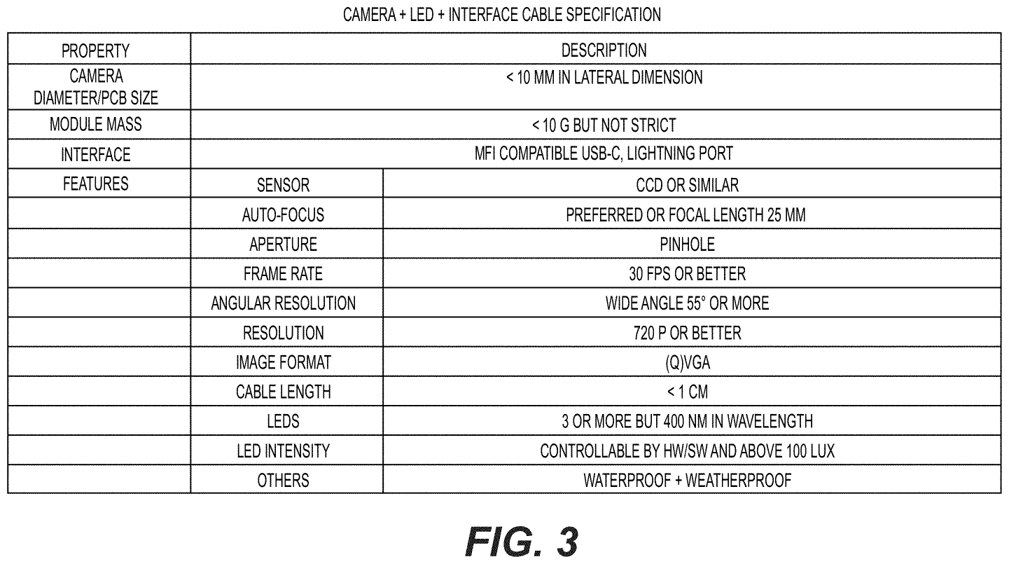

FIG. 3 outlines the components of an example implementation of the data reader. The data reader may be included as part of a single use test kit or as part of a package of multiple test kits. The data reader can be designed to be reused for multiple tests (unlike many existing test readers). The data reader includes a housing, internal camera, internal LEDs for illuminating the test strip when inserted into the data reader, and an interface cable and adapter for connecting to one or more types of smartphones or tablets.

In alternative implementations, a camera sensor on a smartphone or tablet can be used to detect the visual pattern on the test strip instead of using a separate data reader. Although potentially more complex because of variations in ambient lighting, angular positioning of the camera sensor relative to the test cassette, differences among camera sensors from various suppliers (e.g., differing spectral sensitivities, focal lengths, and color gamuts), and the like, the app can be programmed to process images detected by the camera sensor to normalize the spectral data received from the camera sensor for further processing of the spectral signature to obtain test results.

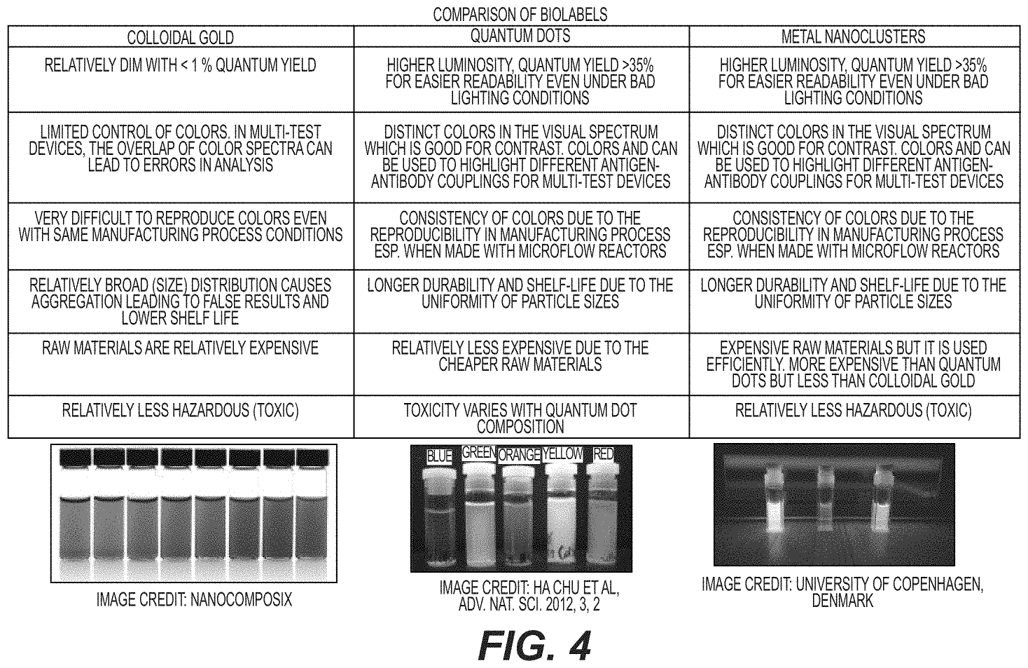

Implementations of the test strip of the diagnostic system can use quantum dots as bio-labels. By replacing the signal marker on the LFIAs from ubiquitously used colloidal gold with quantum dots, the sensitivity and specificity can be improved significantly [B. Liu et al, medRxiv, July 2020][J. Wang et al, ACS Omega 2019, 4, 6789-6795][D. Wang, Nature Biomedical Engineering, 2020, 5, 1150-1158]. Quantum dots also help achieve high performance. While colloidal gold nanoparticles are relatively large (>25 nm in size) biolabels used in sensing, the diagnostic system described in this specification can use novel biolabels (e.g., quantum dots and metal nanoclusters) that are much smaller (<5 nm). These biolabels can therefore, on an equivalent volume basis, offer more surface area. When conjugated with sensory proteins (i.e., antibodies), these biolabels can offer 3 orders of magnitude more binding sites for the analyte proteins (antibodies), which leads to highly improved sensitivity. The novel biolabels also offer various advantages such as higher luminosity, a gamut of distinct colors for multiplexing tests, consistency in manufacturing, longer shelf life, and orders of magnitude cost savings over colloidal gold. FIG. 4 outlines differences between colloidal gold, quantum dots, and metal nanocluster biolabels.

Fluorescent nanoparticle labeled LFIAs have higher sensitivity and allow for in-situ monitoring compared with LFIAs that use colloidal gold (CG) for the bio-labels. [J. Wang et al, ACS Omega 2019, 4, 6789-6795] LFIAs labeled with fluorescent nanoparticles (e.g., quantum dots or fluorescent nanoclusters) have high quantum yields >35%, which enhances readability. In particular, test lines are easier to read and maintain readability much longer than CG-based LFIAs, which have low luminosity resulting from quantum yields <1%, making test lines difficult to read and which causes them to grow more faint with time. Fluorescent nanoparticle-labeled LFIAs also have a wide color gamut (potentially approaching or exceeding a million colors), which makes multiple analyte testing (multiplexing) possible. The limited color gamut (i.e., red and purple) of CG-based LFIAs makes multiplexing difficult. Furthermore, fluorescent nanoparticle-labeled LFIAs have a higher inherent stability over gold, which enhances durability, manufacturing consistency and shelf life of the LFIAs, and a lower cost of manufacture as compared to CG LFIAs.

The fluorescent nanoparticle labeled LFIAs can use quantum dot (such as CdSe/CdS tetrapod quantum dots)/metal (such as Ag) nanocluster technology that requires special manufacturing equipment (e.g., a microflow reactor) that prevents production of counterfeit test strips (i.e., because the quantum dot composition cannot be duplicated). The data reader can include a spectrometer for accurately detecting spectral signatures of the quantum dots and can be tuned to be sensitive only to quantum dots that produce specific spectral responses expected from the authorized quantum dots or can provide the spectral information for software analysis (e.g., by the app). The software in the app can be tuned to analyze the spectral information received from the data reader to be able to distinguish counterfeit test strips from authentic test strips. Thus, the test strips avoid problems with existing diagnostic tests, in which similar looking alternatives can be used to produce counterfeit test strips, as most of the components are readily available worldwide with little or no differentiation.

In certain embodiments, the invention includes multicolor optical coding for biological assays by embedding quantum dots into mesoporous and macroporous beads at precisely controlled ratios. Owing to their novel optical properties such as size-tunable emission and simultaneous excitation, quantum dots are ideal fluorophores for wavelength-and-intensity multiplexing. Kinetics study reveals that quantum dot doping of porous silica and polystyrene beads can be completed from seconds to minutes. Imaging and spectroscopic measurements indicate that the quantum dot-tagged beads are highly uniform and reproducible, yielding bead identification accuracies as high as 99.99% under favorable conditions. Hybridization studies demonstrate that the coding and target signals can be simultaneously read at the single-bead level. This spectral coding technology is expected to open new opportunities in gene expression studies, high-throughput screening, and medical diagnostics.

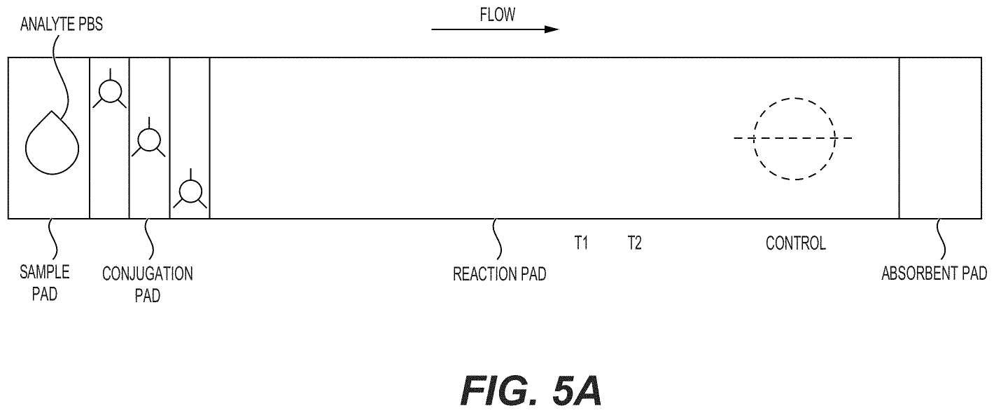

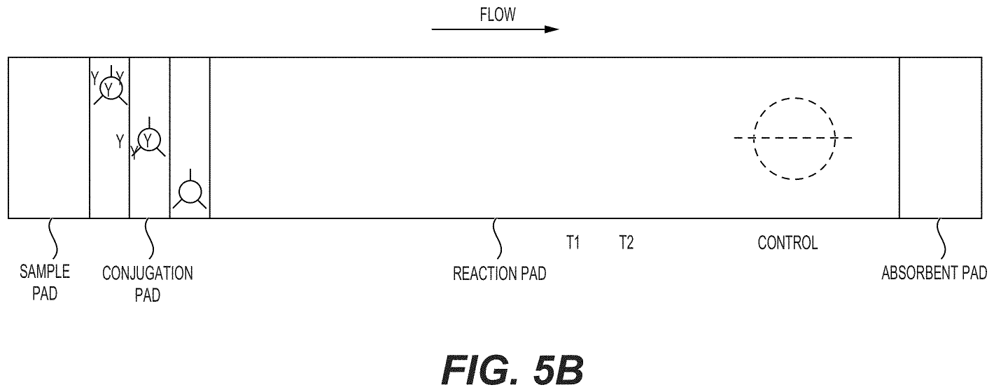

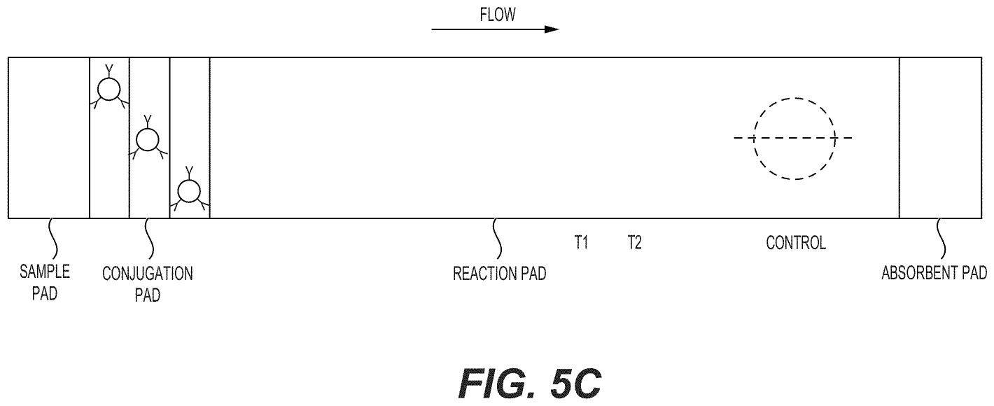

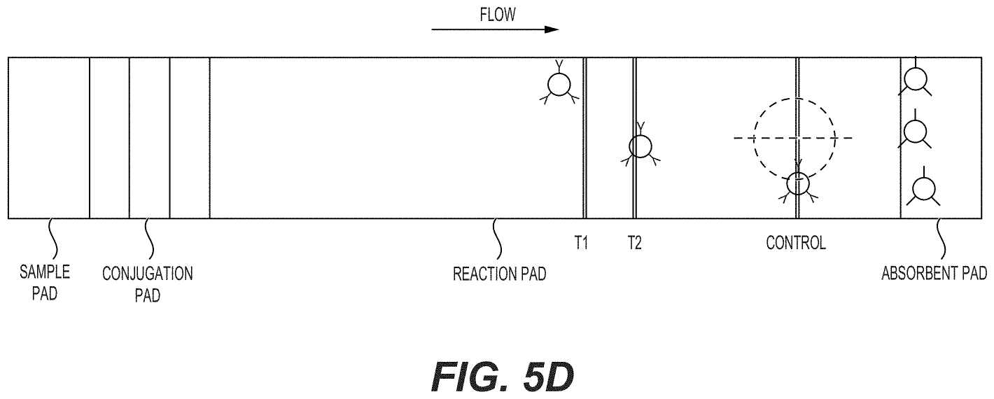

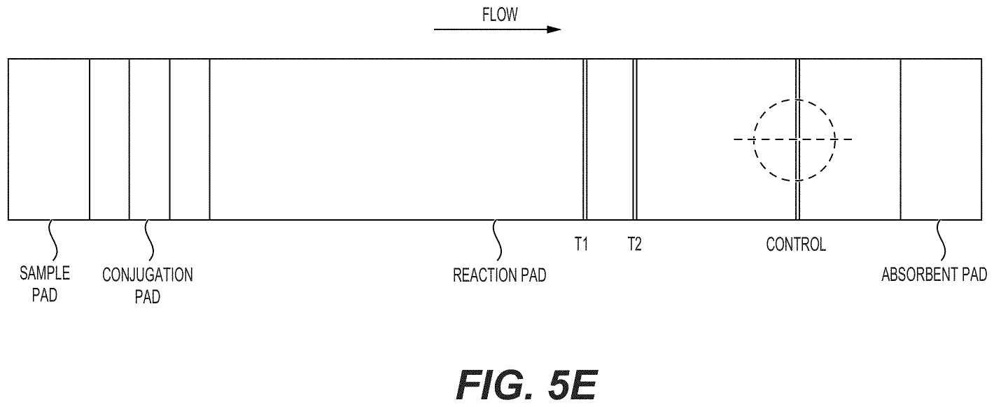

FIGS. 5A-5E illustrate the principle of operation of one implementation of the diagnostic system. An analyte (e.g., blood, blood plasma, saliva, or other bodily fluid) of appropriate concentration is introduced along with a phosphate buffer solution (PBS) as a drop on the sample pad, as shown in FIG. 5A. Antigens 1. Y, 2. Y & 3. Y corresponding to 3 different diseases are transported to the conjugation pad, as depicted in FIG. 5B, and onwards laterally due to capillary action. The antigens 1. Y, 2. Y & 3. Y bind to the corresponding secondary antibodies 1. I, 2. I & 3. I conjugated on biolabels 1. O, 2. O & 3. O, as shown in FIG. 5C. The assembly of antibody+antigen+QD (quantum dots) are collectively transported through the reaction pad and to the corresponding test line 1. T1, 2. T2, 3. C, where they bind with immobilized primary antibodies and form a single bright colored band (e.g., to indicate a positive test result corresponding to the test line), as shown in FIG. 5D. The control line could be designed to indicate presence of lysozymes. Unbound antibody+biolabels flow all the way to the absorbent pad. The test and control lines maintain a visually detectable indication of the test results, as depicted in FIG. 5E.

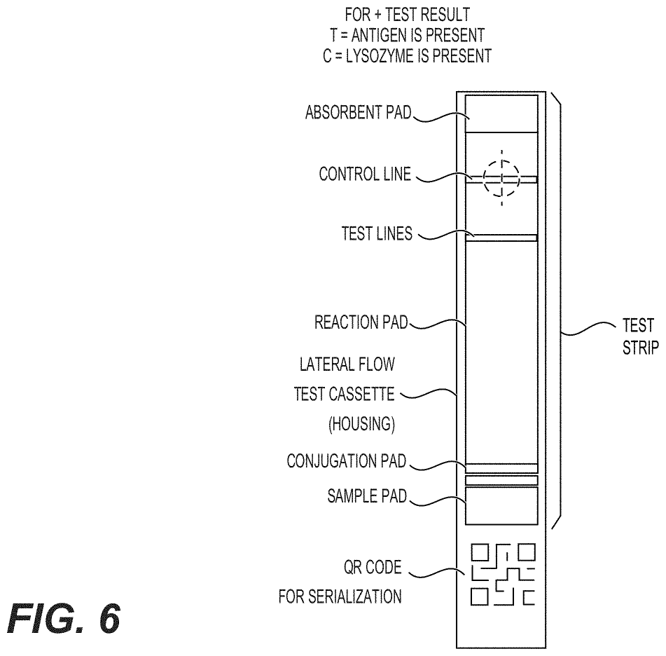

FIG. 6 depicts the lateral flow test cassette and test strip. LFIAs typically have a control line whose function is to indicate that lateral flow has occurred. The test strip of the modular diagnostic system described in this specification uses a control line that confirms lateral flow but that also functions as an indicator for the amount of analyte (blood, saliva, sputum) introduced into the test cassette. In particular, the control line can be used to confirm the presence of matrix in the analyte for inferring that the sample includes a sufficient amount of test molecules (e.g., test molecule 1, test molecule 2, test molecule 3, etc., if present in the sample) to activate the corresponding test lines (e.g., test line 1, test line 2, test line 3, etc.). This is useful for testers to perform the test without the help of a medical professional and to eliminate misuse such as using neutralized saliva (e.g., after consuming coffee). Current generation test strips have a control line which lights up only when the free gold nanoparticles flow to and immobilize on it. In a lit condition, it confirms for presence of buffer, functioning of the nanoparticles and the flow of both. But it does not account for the absence of sample (analyte) or insufficient analyte. If sufficient saliva is not drawn using a cheek swab then the test strip might still display a result but it will not be a representation of the tested person's medical condition. In the test strip described in this specification, a novel control line tests for amylase/lipase activity in the analyte introduced on the test strip. This control line will not light up if there is insufficient saliva introduced on the test strip or sufficient but neutralized saliva (e.g., because of coffee, mouthwash, soda, etc.).

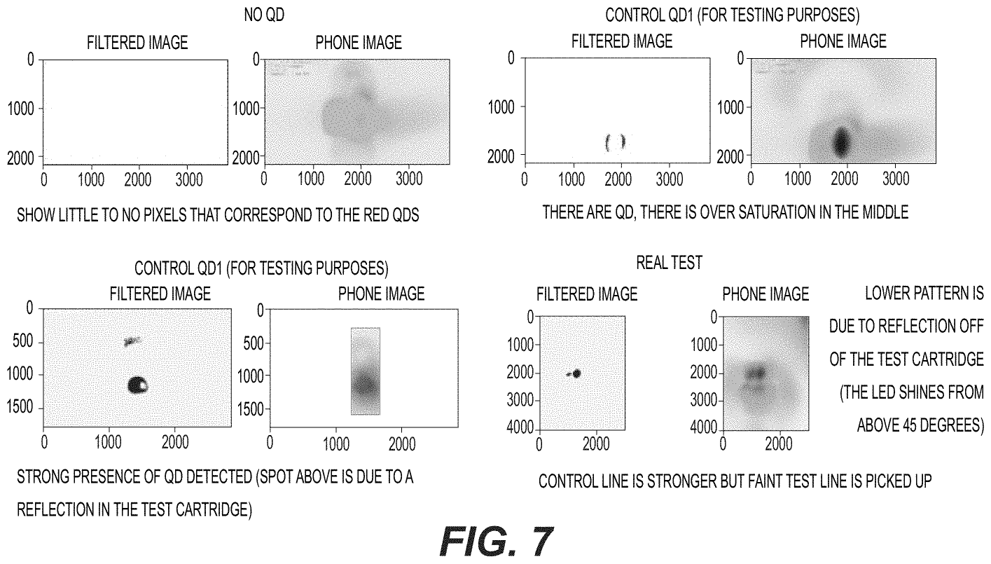

FIG. 7 depicts filtered images and images detected by a smartphone when quantum dots are or are not detected at a control line and on a test line.

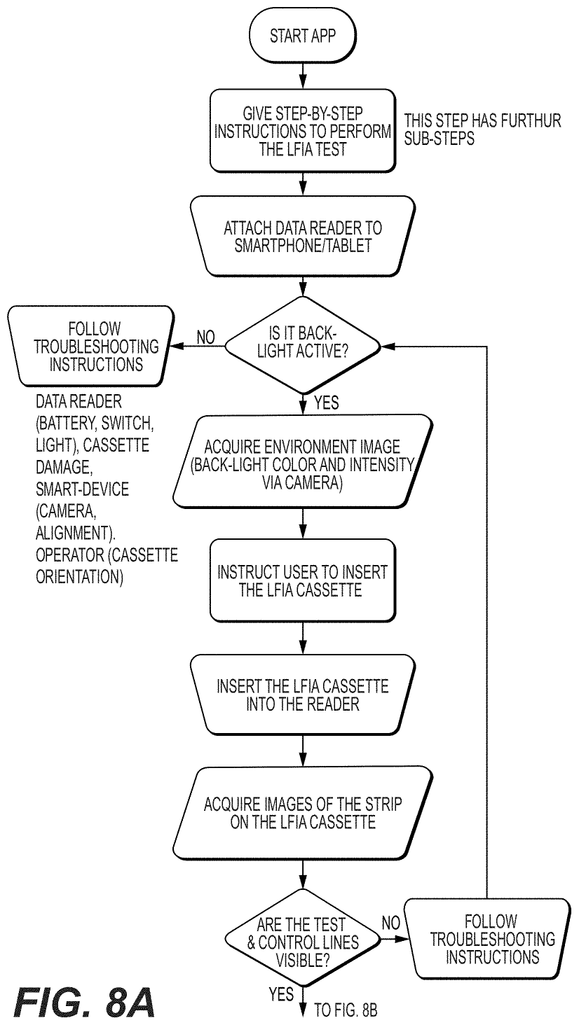

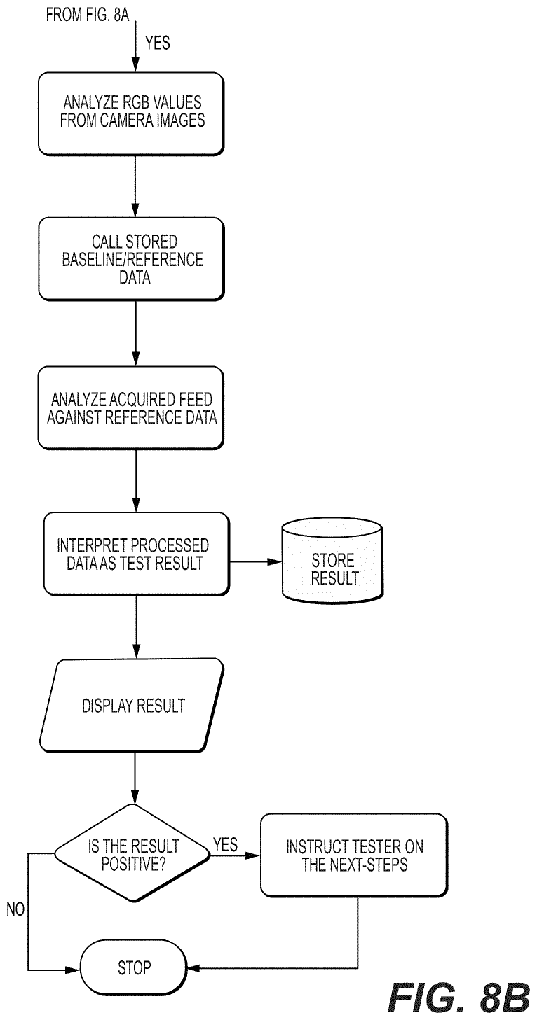

FIGS. 8A and 8B are a flow diagram of the process of using the app. After staring the app, the user/tester is given step-by-step instructions on how to perform the LFIA test. The user attaches the data reader to the smartphone/tablet. If the backlight of the data reader is not active, the user is presented with troubleshooting instructions. Otherwise, an environmental image (including the backlight color and intensity) is acquired via the data reader camera The user is instructed by the app to insert the LFIA test cassette into the data reader, and, after the test cassette is inserted, images of the test strip on the LFIA cassette are acquired by the data reader camera. If the test lines and control are not visible, the user is presented with troubleshooting instructions. Otherwise, RGB values from the camera images are analyzed by the app. The app also retrieves stored baseline and/or reference data, and processes or compares the analyzed RGB values to the baseline and/or reference data. The processed data is interpreted as a test result and stored in a database. The result is also displayed by the app, and, if the test result is positive, the user is instructed on next steps.

The modular diagnostic system can also enable use by testers who are not skilled in reading the lateral flow test, such as use in the home and by untrained users. Current LFIAs can require the tester to be skilled in reading the lateral flow test or to learn the skill by reading a manual. The modular diagnostic system includes a user-friendly app on a smartphone to guide the tester and a data reader device with electronics optimized to perform and analyze the lateral flow test preventing improper use of the test and loss of test result integrity. For example, a slot in the data reader can be shaped to prevent improper insertion of the test cassette by allowing insertion only in the proper orientation and the app can provide step-by-step instructions with graphics to guide the tester throughout the testing process.

In some implementations, the diagnostic system can be configured to offer covert testing capabilities, which prevents or limits an average tester's ability (and at least makes it more difficult for an expert in the art) to read the result of the test. Such covert testing can prevent faking of test results. Covert technology can be implemented by developing unique color-coded test strips which do not present results in the same manner as other test strips, and therefore it is not possible to deduce the outcome of the result of one LFIA based on the results of another LFIA. In particular, the quantum dots can be selected for different test strips such that the spectral response corresponding to a positive test result, for example, may be substantially or somewhat different between different test strips. The spectral response corresponding to a positive, negative, or multiplexed test result for a particular test strip can be encoded on the test cassette, which can be read by the data reader and decoded only by persons with authorized access to the decoding algorithm. For example, a key to reading the test strips can be decoded using a machine-readable (QR) code on the test cassette. Alternatively, the spectral response data and the information encoded on the test cassette may require decoding by a remote software platform, which can receive serialized spectral response data and QR code data, validate the test cassette, and interpret the test results based on the received data.

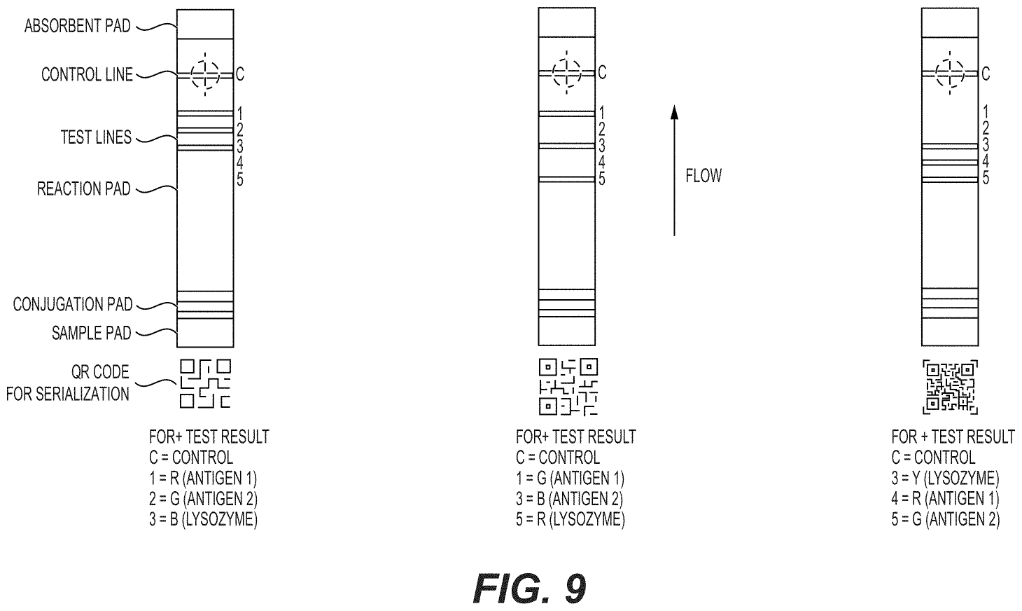

FIG. 9 depicts examples of different configurations of test strips implementing a covert testing capability. In each case, the result (positive/negative) from the test strip is construed from the test lines on the membrane and the QR code on the test cassette. The test lines are used to detect the presence of an antigen, while the control line is used to detect lysozymes, which are enzymes that are omnipresent in saliva. The test lines and QR code are known and placed by the manufacturer and may be at different locations and have different colors representing the same result. For example, the QR code in the first test strip can be decoded to reveal that the presence of the color red at line 1 indicates that a first antigen is present in the sample, the color green at line 2 indicates that a second antigen is present, and the color blue at line 3 indicates that a lysozyme is present. The second test strip reveals the same results when the color green is present at line 1, blue at line 3, and red at line 5, and the third test strip reveals the same results when the color red is present at line 4, the color green is present at line 5, and the color yellow is present at line 3. In each case, the QR code can be used to decode what patterns correspond to positive test results. As discussed above, the lysozyme can be used to confirm that the sample is not neutralized. In this example, the control line may be used to detect whether a sufficient amount of sample is present.

The test lines may further use a specific color or spectral pattern to indicate a positive or negative test result, such that the mere presence of a visible line cannot be interpreted as a positive or negative result. Rather, the result is only interpreted as a positive test result if a specific spectral pattern is detected.

The diagnostic system is economical and scalable to 10⁹/year scale or more. The smartphone/tablets can be provided by the tester and are ubiquitous. The app for the smartphone/tablets can be downloaded from the popular app stores. The data reader is made using inexpensive/recyclable/biodegradable plastic and its components (camera module, LED light source, and spectrometer) are cheap to build/source. Finally, the lateral flow test cassettes are already being built economically in the billion unit/year scale.

The diagnostic system can also be made to be disposable and biodegradable. Besides the smartphone/tablet and app module of the diagnostic system, the other two modules are disposable and biodegradable. Biodegradable plastic can be used for the test cassette and data reader housing. The test strip in the test cassette uses biodegradable components except for the biolabels which, if made using non-hazardous quantum dots or metal clusters (or an acceptably low quantity of potentially hazardous quantum dots or metal nanoclusters), can be disposable. The data reader components (camera and LED light source) can also be made to be certified as disposable in household waste.

While this specification contains many implementation details, these should not be construed as limitations on the scope of the invention or of what may be claimed, but rather as descriptions of features specific to particular implementations of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

In various embodiments, POC rapid diagnostics of the invention use the LFIA platform. The LFIA platform is extremely versatile. In various embodiments, the detection of high-molecular-weight antigens requires an antibody pair where an antibody to one analyte epitope is labeled with a reporter, such as colloidal gold or quantum dots, and a capture antibody to a second epitope on the same analyte is immobilized on the lateral flow strip. In various embodiments, an antigen-capture sandwich format, the intensity of the signal at the test line is proportional to the concentration of the analyte. In various embodiments, the sandwich immunoassays are POC tests for infectious diseases that detect microbial products in clinical samples, e.g., the group A streptococcal cell wall carbohydrate. In various embodiments, the detection of low-molecular-weight analytes with a single antigenic determinant requires a competitive format. In these assays, the intensity of the test line is inversely proportional to the analyte concentration. Examples of assays using competitive formats include many immunoassays for the detection of drugs of abuse. In various embodiments, the LFIA format can be used for the detection of subject antibodies to target antigens. In this instance, the target antigen is immobilized on the strip, and the binding of patient antibody is detected by the use of a labeled reporter, such as a second antibody. Examples of serological assays in the LFIA format include tests for HIV-1/2 or hepatitis C virus.

In various embodiments, the performance of LFIA of the invention for antigen detection is dependent on the concentration of the analyte in a clinical sample. Analyte concentrations below the assay limit of detection for the test may produce a false-negative result.

### EXAMPLE

Serum samples are obtained from patients with COVID-19 at different points during the disease course. The samples are used for the evaluation of a POC rapid test for detection of anti- SARS-CoV-2 antibodies.

Serum samples, IgM antibody is detected in samples using the claimed 2019-nCoV IgG/IgM Rapid Test. IgG antibody is also detected in samples using the 2019-nCoV IgG/IgM Rapid Test. Presence of either IgG or IgM is detected in samples using the 2019-nCoV IgG/IgM Rapid Test.

The antibody responses at different time points during the disease course after symptom onset are further evaluated using the found rapid tests. Anti-SARS-CoV-2 antibody is detected in 100% serum samples collected after 3 weeks of symptom onset using all rapid tests. The 2019-nCoV IgG/IgM Rapid Test detects high percentage and long duration of IgM in serum samples.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A system comprising:
a lateral flow immunochromatographic assay (LFIA) test cassette having at least one of quantum dot or metal nanocluster biolabels; and
a software application adapted to interpret test results from detected images of the LFIA test cassette.

2. The system of claim 1 wherein test results are decoded based on data coded on a housing of the test cassette.

3. A system comprising:
a lateral flow immunochromatographic assay (LFIA) test cassette having at least one of quantum dot or metal nanocluster biolabels; and
a data reader adapted to receive LFIA test cassette and detect images on a test strip of the LFIA test cassette for use in interpreting test results.

4. A system comprising:
a lateral flow immunochromatographic assay (LFIA) test cassette having at least one of quantum dot or metal nanocluster biolabels, wherein the LFIA test cassette includes a control line adapted to detect presence of a lysozyme.

5. A real-time, point of care diagnostic for identifying the presence of antibodies to the SARS-CoV-2 virus in a sample, comprising in combination:
(a) a lateral flow immunoassay test device including a strip having an axis and having a control line and two test lines oriented perpendicular to the axis, the two test lines changing color in the presence of IgG and IgM antibodies to the SARS-CoV-2 virus in the sample, respectively, the test device receiving the sample, and a reader for the lateral flow immunoassay testing device,
(b) wherein the test strip comprises quantum dot labels;
(c) wherein the reader comprises a housing enclosing electronics and an optics unit configured and positioned within the reader for reading the test lines and the control line of the test device;
(d) a processing unit using a reading of the optical calibration test pattern by the said optics unit to perform a self-test of the optics unit and correction for any nonlinearity of the electronics or optics unit, wherein the optical calibration test pattern and the test lines and the control line are read sequentially by the optics unit upon movement of the tray from the open position to the closed position,
(e) the processing unit further generating a result for the test device based on the reading of the test lines and control line by the optics unit indicating whether antibodies to the SARS-CoV-2 virus are present in the sample.
